(19) United States
(12) Patent Application Publication
(10) Pub. No.: US 2022/0143261 A1
(43) Pub. Date: May 12, 2022

(54) PHOTOCATALYST AND PREPARATION METHOD THEREFOR

(71) Applicant: JCHI Global, Yeonsu-gu, Incheon (KR)

(72) Inventor: Young Dok KIM, Suwon-si, Gyeonggi-do (KR)

(21) Appl. No.: 17/284,774

(22) PCT Filed: Oct. 25, 2019
(86) PCT No.: PCT/KR2019/014146

§ 371 Date: Apr. 12, 2021

(30) Foreign Application Priority Data
Oct. 26, 2018 (KR) 10-2018-0129020
Jun. 28, 2019 (KR) 10-2019-0077668
Jun. 28, 2019 (KR) 10-2019-0077669
Jun. 28, 2019 (KR) 10-2019-0077670
Jun. 28, 2019 (KR) 10-2019-0077671

(51) Int. Cl. A61L 9/20; B01J 23/745; B01J 21/06; B01J 35/00; B01J 37/08; B01J 35/10; B01J 35/02; C09D 5/14; C09D 7/62; C23C 16/40; C23C 16/56

(52) U.S. Cl. CPC A61L 9/205; B01J 23/745; B01J 21/063; B01J 35/004; B01J 35/0006; B01J 37/082; C23C 16/56; B01J 35/1061; B01J 35/1057; B01J 35/023; C09D 5/14; C09D 7/62; C23C 16/406; B01J 35/1009

(57) ABSTRACT

A photocatalyst, a product including a photocatalyst, and a method for preparing a photocatalyst are provided. The photocatalyst is an inorganic oxide-based photocatalyst including inorganic oxide and a ferrocene-derived iron oxide layer formed on the inorganic oxide.

### TECHNICAL FIELD

The present invention relates to a photocatalyst with an expanded catalytic active wavelength range and a method for preparing the same.

### BACKGROUND ART

Photocatalysts have catalytic activity by absorbing light energy, and oxidatively decompose environmental pollutants such as organic substances with strong oxidizing power by catalytic activity. Namely, the photocatalysts irradiate a light having a bandgap or higher energy (ultraviolet rays) to cause a transition of electrons from the valence band to the conduction band and to form holes in the valence band. These electrons and holes diffuse to the surface of the powder, and are brought into contact with oxygen and moisture to cause an oxidation-reduction reaction, or recombined to generate heat. In other words, the electrons of the conduction band reduce oxygen to produce superoxide anions, and the holes of the valence band oxidize moisture to form hydroxy radicals (OH·). The photocatalysts may show decomposition, sterilizing power, hydrophilicity, etc. of organic matters in a gas or liquid phase adsorbed on the surface of the photocatalysts, i.e., recalcitrant organic matters by strong oxidizing power of the hydroxy radicals (OH·) formed by these holes. In general, a titanium dioxide (TiO₂) powder is used as a photocatalyst, and titanium dioxide (TiO₂) has advantages that it is harmless to the human body, has excellent photocatalytic activity, has excellent light corrosion resistance, and is inexpensive. Titanium dioxide (TiO₂) generates electrons (conduction band) and holes (valence band) by absorbing and reacting with ultraviolet rays of 388 nm or less. At this time, the ultraviolet rays used as a light source may include artificial lighting such as lamps, incandescent lamps, and mercury lamps, light-emitting diodes in addition to sunlight. The electrons and holes generated in the reaction are recombined in a 10⁻¹² to 10⁻⁹ second, but if contaminants or others are adsorbed on the surface before the recombination, the contaminants or others are decomposed by the electrons and holes. However, since about 2% of the light may be used in obtaining the bandgap energy (wavelength of 380 nm or more) of the titanium dioxide (TiO₂) powder from sunlight, it is difficult to have smooth catalytic activity in the visible light region (400 to 800 nm) which is a main wavelength of sunlight. That is, in order to respond to visible light, it is essential to effectively reduce the bandgap of the photocatalyst and efficiently separate pairs of electrons/holes generated through light absorption, but the efficiency in a visible light-responsive photocatalyst of the titanium dioxide (TiO₂) powder has not yet reached the level for commercialization in the air cleaning field.

### DISCLOSURE

### Technical Problem

The present invention is to solve the above-mentioned problem, and the present invention is to provide an inorganic oxide-based photocatalyst having excellent photocatalytic activity in the visible light region, formed by introducing a ferrocene doping process.

The present invention is to provide a photocatalyst composition including an inorganic oxide-based photocatalyst according to the present invention.

The present invention is to provide a product including an inorganic oxide-based photocatalyst according to the present invention and having a photolysis function.

The present invention is to provide a method for preparing an inorganic oxide-based photocatalyst according to the present invention using a ferrocene doping process.

However, tasks to be solved by the present invention are not limited to those mentioned above, and other tasks which have not been mentioned will be clearly understood by those skilled in the art from the following description.

### Technical Solution

The present invention relates to an inorganic oxide-based photocatalyst, and the photocatalyst according to an embodiment of the present invention may include an inorganic oxide and an organometallic compound-derived metal oxide layer.

According to an embodiment of the present invention, the present invention relates to an inorganic oxide-based photocatalyst including: an inorganic oxide; and a ferrocene-derived iron oxide layer formed on the inorganic oxide.

According to an embodiment of the present invention, iron may be contained in the ferrocene-derived iron oxide layer in an amount of 0.001 to 10 wt % compared to the inorganic oxide.

According to an embodiment of the present invention, the ferrocene-derived iron oxide layer may be obtained by performing a heat treatment process on ferrocene deposited on the inorganic oxide.

According to an embodiment of the present invention, the inorganic oxide may include at least one selected from the group consisting of oxides including at least one of Ti, Zn, Al, and Sn.

According to an embodiment of the present invention, the inorganic oxide may include at least one selected from the group consisting of a bead form, a powder form, a rod form, a wire form, a needle form, and a fiber form, and the inorganic oxide may have a size of 1 nm to 500 μm.

According to an embodiment of the present invention, the inorganic oxide-based photocatalyst may have photoactivity in a visible light region of 400 nm or more, and the inorganic oxide-based photocatalyst may have photoactivity in a dry condition of 30% or less humidity.

According to an embodiment of the present invention, the ferrocene-derived iron oxide may include one or more of compounds represented by the following Chemical Formula 1:

FeₓO_{Y}H_{Z} [Chemical Formula 1]
(X, Y, and Z are each selected from 0 to 3, and X and Y are not 0.)

According to an embodiment of the present invention, the inorganic oxide-based photocatalyst may have a specific surface area of 5 (m²/g) or more.

The present invention relates to a photocatalyst composition including the inorganic oxide-based photocatalyst of claim 1 according to an embodiment of the present invention, in which the photocatalyst may be contained in an amount of 0.01 to 99 wt % in the photocatalyst composition.

The present invention relates to a product having a photolysis function, including the inorganic oxide-based photocatalyst of claim 1 according to an embodiment of the present invention.

According to an embodiment of the present invention, the product may be a molded body in which the inorganic oxide-based photocatalyst is coated on a substrate, or which includes the inorganic oxide-based photocatalyst.

According to an embodiment of the present invention, the product may be applied to building materials for air purification having a function of photolyzing acidic gases and organic sub stances.

According to an embodiment of the present invention, the building materials for air purification may be one or more of paints, wallpapers, blinds, sidewalk blocks, median separators, artificial turf, artificial turf filler, elastic pavement materials, flooring materials, asphalt, concrete, and elastic mats.

According to an embodiment of the present invention, the present invention relates to a method for preparing an inorganic oxide-based photocatalyst, the method including the steps of: preparing an inorganic oxide; forming a ferrocene layer on the inorganic oxide; and forming a ferrocene-derived iron oxide layer by performing a heat treatment process after the step of forming the ferrocene layer.

According to an embodiment of the present invention, the step of forming the ferrocene layer may be performed by using a wet coating method, a sputtering method, or a deposition method, the step of forming the ferrocene layer may be carried out at room temperature to 120° C., and the ferrocene layer may include 0.001 to 20 wt % of ferrocene compared to the inorganic oxide.

According to an embodiment of the present invention, the step of forming the ferrocene layer may be forming a ferrocene deposition layer using temperature-regulated chemical vapor deposition (TR-CVD).

According to an embodiment of the present invention, the step of forming the ferrocene-derived iron oxide layer may include the steps of: performing a first heat treatment process at a temperature of 100 to 300° C.; and performing a second heat treatment process at a temperature of 300 to 900° C., in which the steps may each include performing the heat treatment process at different temperatures.

### Advantageous Effects

The present invention may provide an inorganic oxide-based photocatalyst having excellent photocatalytic activity in the visible light region and excellent photolysis efficiency in various humidity and temperature ranges.

The present invention may provide an inorganic oxide-based photocatalyst in a simple and economical way, and the inorganic oxide-based photocatalyst may be effectively applied as a material for indoor and outdoor air purification by having excellent stability and the ability to decompose volatile organic compounds with high efficiency in response to light in the visible light region.

The present invention provides an inorganic oxide-based photocatalyst having excellent photocatalytic activity in the visible light region by the ferrocene doping process, and the photocatalyst may be applied to building materials, interior accessories, etc. that can add and utilize the photoactive function.

### DESCRIPTION OF DRAWINGS

FIG. 1 shows a flowchart of a method for preparing an inorganic oxide-based photocatalyst according to the present invention, according to an embodiment of the present invention.

FIG. 2 exemplarily shows the configuration of a TR-CVD reactor used in the preparation process of the inorganic oxide-based photocatalyst according to the present invention, according to Examples of the present invention.

FIG. 3 exemplarily shows the preparation process of the inorganic oxide-based photocatalyst according to the present invention, according to Examples of the present invention.

FIG. 4 shows an image of inorganic oxide-based photocatalysts prepared according to Examples of the present invention.

FIG. 5 shows transmission electron microscope (TEM) images of the inorganic oxide-based photocatalysts prepared according to Examples of the present invention.

FIG. 6 shows evaluation results of the photolysis performance of the inorganic oxide-based photocatalysts prepared according to Examples of the present invention.

FIG. 7 shows evaluation results of the photolysis performance according to the humidity of the inorganic oxide-based photocatalysts prepared according to Examples of the present invention.

FIG. 8 shows stability evaluation results of the photolysis performance according to repeated photolysis experiments of the inorganic oxide-based photocatalysts prepared according to Examples of the present invention.

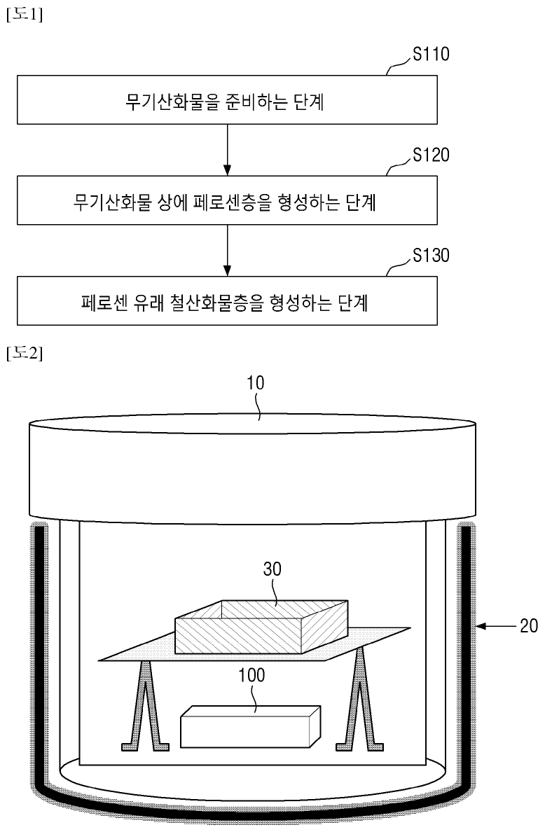

The present invention may relate to a photocatalyst, a product including the same, and a method for preparing the photocatalyst, and more specifically, to an inorganic oxide-based photocatalyst, a product including the same, and a method for preparing the photocatalyst, the inorganic oxide-based photocatalyst including: an inorganic oxide; and a ferrocene-derived iron oxide layer formed on the inorganic oxide.

### DETAILED DESCRIPTION OF EMBODIMENT

Hereinafter, embodiments of the present invention will be described in detail with reference to the accompanying drawings. In the description of the present invention, if detailed descriptions of related disclosed art or configuration are determined to unnecessarily make the gist of the present invention obscure, they will be omitted. Also, terms used in the present specification, as terms which are used so as to appropriately describe a preferred embodiment of the present invention, might be differently defined depending on the user's or operator's intention or the practices of the field that the present invention belongs to. Therefore, the terms should be defined based on overall contents of the present specification. The same reference numerals as shown in each drawing represent the same elements.

Throughout the present specification, when any member is positioned “on” the other member, this not only includes a case that any member is brought into contact with the other member, but also includes a case that another member exists between two members.

Throughout the present specification, if a prescribed part “includes” a prescribed element, this means that another element can be further included instead of excluding other elements.

Hereinafter, an inorganic oxide-based photocatalyst according to the present invention will be described in detail with reference to Examples and drawings. However, the present invention is not limited to such Examples and drawings.

The present invention relates to an inorganic oxide-based photocatalyst, and the photocatalyst according to an embodiment of the present invention may include an inorganic oxide and an organometallic compound-derived metal oxide layer.

The present invention relates to an inorganic oxide-based photocatalyst, and the photocatalyst according to an embodiment of the present invention may include an inorganic oxide and a ferrocene-derived iron oxide layer formed by a ferrocene doping process. The ferrocene-derived iron oxide layer may be formed as a coating layer on the inorganic oxide, and may improve light absorption and photocatalytic efficiency in the visible light region.

The inorganic oxide is an inorganic semiconductor compound that absorbs light energy and exhibits catalytic activity. For example, the inorganic oxide may be an oxide including at least one selected from the group consisting of Ti, Zn, Al, Fe, W, Sn, Bi, Ta, Cu, Si, Ru, Sr, Ba and Ce, preferably Ti, Zn, Al, and Sn. Specifically, the inorganic oxide may be TiO₂, Al₂O₃, ZnO₂, ZnO, SrTiO₃, Fe₂O₃, Ta₂O₅, WO₃, SnO₂, Bi₂O₃, NiO, Cu₂O, SiO, SiO₂, MoS₂, InPb, RuO₂, CeO₂, or the like. Further, the inorganic oxide may further include semiconductor compounds such as CdS, GaP, InP, GaAs, and InPb in addition to the oxides.

The inorganic oxide may include at least one form selected from the group consisting of a bead form, a powder form, a rod form, a wire form, a needle form, and a fiber form, and the inorganic oxide may have a size of: 1 nm or more; 10 nm or more; 30 nm to 500 μm; 30 nm to 100 μm; or 30 nm to 1 μm. The size may mean diameter, thickness, length, etc. depending on the form.

The ferrocene-derived iron oxide layer is formed by a ferrocene doping process. For example, a ferrocene layer formed on the inorganic oxide is heat-treated to pyrolyze ferrocene, and the ferrocene-derived iron oxide layer may include an iron oxide which is converted from ferrocene by this pyrolysis process. The ferrocene doping process will be described more specifically in the following preparation method.

The ferrocene-derived iron oxide is an iron oxide derived from at least one of ferrocene and ferrocene derivatives, and the ferrocene derivatives may include at least one selected from the group consisting of ferrocene aldehyde, ferrocene ketone, ferrocene carboxylic acid, ferrocene alcohol, a phenol or ether compound, a nitrogen-containing ferrocene compound, a sulfur-containing ferrocene compound, a phosphorus-containing ferrocene compound, a silicon-containing ferrocene compound, 1,1′-di-copper ferrocene, ferrocene boric acid, ferrocenyl cuprous acetylide, and bisferrocenyl titanocene.

Iron may be contained in the ferrocene-derived iron oxide layer in an amount range of: 0.001 to 10 wt %; 0.01 to 10 wt %; 0.01 to 3 wt %; 0.001 to 5 wt %; 0.001 to 6 wt %; 0.01 to 1.5 wt %; or 0.01 to 1 wt % compared to the inorganic oxide. If iron is contained in the ferrocene-derived iron oxide layer within the ranges, photolysis efficiency may be improved by increasing photocatalytic activity in the visible light region. In addition, although the absorption of the visible light region may increase if the iron content increases, the photocatalytic activity may decrease due to such an increase in the iron content. Therefore, it may be preferable to contain iron within the above-mentioned ranges, and it may be more preferable to contain iron in an amount range of 0.01 to 1 wt %.

The ferrocene-derived iron oxide layer may have a thickness range of: 0.01 nm or more; 0.1 nm or more; 10 nm or more; or 1 to 100 nm. When the thickness of the ferrocene-derived iron oxide layer is included within the thickness range, it is possible to improve the photolysis performance by preventing a decrease in the porosity of a photocatalyst due to an increase in the thickness of the coating layer, and increasing the adsorption amount of moisture, OH-ions, a decomposition target, or the like on the surface. Further, the ferrocene-derived iron oxide layer may include a ferrocene-derived iron oxide having a size of: 0.01 nm or more; 0.1 nm or more; 10 nm or more; or 1 to 100 nm. The size may mean length, diameter, thickness, etc. depending on the form.

The ferrocene-derived iron oxide may include one or more of compounds represented by the following Chemical Formula 1:

FeₓO_{Y}H_{Z} Chemical Formula [1]

where, X, Y, and Z are each selected from 0 to 3, and X and Y are not 0. That is, the ferrocene-derived iron oxide may form a photocatalyst which responds to the visible light by introducing iron oxide (FeₓO_{y}H_{z}), a semiconducting material that absorbs light of the visible light region, and is stable and inexpensive, to the TiO₂ surface in the form of nano-sized particles.

According to an embodiment of the present invention, the inorganic oxide-based photocatalyst may expand a wavelength range that exhibits a photoreaction by light absorption from an ultraviolet light region to a visible light region, and may exhibit excellent photocatalytic activity, especially in a visible light region of 400 nm or more. In addition, the inorganic oxide-based photocatalyst may have photocatalytic activity in various humidity ranges and may exhibit excellent photocatalytic activity even under dry conditions with a humidity of 30% or less by improving the photocatalytic reactivity that can adsorb and decompose a decomposition target on the surface.

According to an embodiment of the present invention, the inorganic oxide-based photocatalyst may have a specific surface area of: 5 (m²/g) or more; 5 (m²/g) to 1,000 (m²/g); or 5 (m²/g) to 100 (m²/g), and an average pore size of 50 nm or less. That is, the inorganic oxide-based photocatalyst may increase the adsorption amount of the decomposition target on the surface of the photocatalyst by introducing a ferrocene-derived iron oxide to the surface, and may improve the efficiency of the photocatalyst by increasing the photolysis reactivity.

According to an embodiment of the present invention, the inorganic oxide-based photocatalyst may be applied to the decomposition of various harmful substances. In other words, the inorganic oxide-based photocatalyst may be used for the treatment of environmental pollutants, odor substances, organic compounds, acidic gases, etc. For example, the inorganic oxide-based photocatalyst may be used for adsorption and/or photolysis of at least one of substances of gas, liquid, and solid, and may exhibit photoactivity by light energy including various rays of halogen lamps, xenon lamps, sunlight, light-emitting diodes, etc. More specifically, the gas may include an acidic gas, a basic gas, volatile organic compounds (VOCs) such as acetaldehyde and ketones, hydrocarbons such as aromatic hydrocarbons and aliphatic hydrocarbons (paraffin-based and olefin-based), an ozone gas, organic and inorganic free gases, etc., and more specifically, it may include carbon dioxide, carbon monoxide, NOₓ, SOₓ, HCl, HF, NH₃, methylamine, formaldehyde, hydrogen sulfide, amine, methyl mercaptan, hydrogen, oxygen, nitrogen, methane, paraffin, olefin, etc. The liquid may include formaldehyde, acetaldehyde, benzene, toluene, methyl ethyl ketone (MEK), trichloroethylene, disinfectant, gasoline, diesel, oil, alcohol, phenol, dye, etc., and the solid may include a transition metal, precious metals such as Pt, Pd, and the like, ions and/or particles of Hg, Cr, and the like, nanoparticles of 100 nm or less, etc. However, the gas, liquid and solid are not limited thereto.

According to an embodiment of the present invention, the present invention relates to a photocatalyst composition including an inorganic oxide-based photocatalyst.

The inorganic oxide-based photocatalyst may be contained in an amount of 0.01 to 99 wt % in the photocatalyst composition.

The photocatalyst composition may include a balance of a water-based solvent, an oil-based solvent, or both thereof, and the water-based solvent, the oil-based solvent, or both thereof may be appropriately selected depending on the application fields. For example, although the water-based solvent and the oil-based solvent may include water, C₁-C₄ lower alcohols such as methanol, ethanol, propanol, isopropanol, and butanol, the water-based solvent and the oil-based solvent are not limited thereto.

The photocatalyst composition, if it does not deviate from the purpose of the present invention, may further include additives depending on the performance improvement and application field, and the additives may further include a surfactant, a siloxane-based binder, an antibacterial agent, a disinfectant, etc., but are not specifically mentioned in the present specification.

The photocatalyst composition may be coated on a substrate, or may be molded in various forms. For example, although the substrate may include one or more selected from the group consisting of: cellulose paper; synthetic wood, wood; fiber; fabric; and metal, polymer resin or glass, and powder, sheet, film or bead of glass, the substrate is not limited thereto. Further, although the substrate may be finished products requiring the photocatalytic function, e.g., lamps, TVs, refrigerators, notebooks computers, home appliances, wallpapers, concrete, interior accessories such as blinders, furniture, tiles, and mats, building materials, etc., the substrate is not limited thereto.

According to an embodiment of the present invention, the present invention relates to a product having the photocatalytic function, including an inorganic oxide-based photocatalyst according to the present invention. The product may also exhibit the air purification function along with the photocatalytic function. For example, the product may have the photolysis function and/or the air purification function by photoactivity of volatile substances, odor substances, pollutants, etc.

According to an embodiment of the present invention, the product may be a molded body in which the photocatalyst may be bound to a substrate by coating, or which may include the photocatalyst.

For example, the product may be a formulation including a photocatalyst or photocatalyst composition-coated substrate, a photocatalyst or photocatalyst composition-impregnated substrate, a photocatalyst or photocatalyst composition-molded substrate, and solid, liquid or both thereof including the photocatalyst or photocatalyst composition. The molding method may include mixing a photocatalyst and a photocatalyst composition according to the present invention with a molding material or allowing the photocatalyst and the photocatalyst composition to be molded by injecting the photocatalyst and the photocatalyst composition together with the molding material during injection molding of the molding material.

For example, although the formulation may include formulations such as powder, solid, suspension, emulsion, cream, ointment, gel, and liquid, and may include, for example, ink, paint, dyeing agent, etc., the formulation is not limited thereto.

For example, although the product may be applied to clothing such as masks, helmets, gas masks, quarantine clothing, and firefighting clothing, pharmaceuticals, cosmetics, sensors, semiconductors, lithium batteries, solar cells, boilers, electronic products such as mobile phones, notebook computers, PCs, refrigerators, air conditioners, heaters, electric pads, and microwave ovens, kitchen appliances such as gas ranges, gas ovens, kettles, spoon and chopsticks, and tableware, furniture such as beds and wardrobes, accessories such as necklaces, interior or building materials such as inks, paints (water-based, oil-based), color paints, dyes used for dyeing agents and the like, artificial turf, artificial turf filler, elastic pavement materials, flooring materials, asphalt, concrete, elastic pavement materials for children's play facilities, sidewalk blocks, median separators, glass, insulation sheets, wallpapers, blinds, windows, mats, elastic mats, yoga mats, and tiles, lighting devices or fixtures such as incandescent or LED lamps, and desk lamps, materials for air purification such as cooling and heating machine filters, dehumidifier filters, and air cleaner filters, and medical equipment and clothing such as thermometers, syringes, stethoscopes, diagnostic kits or patches, and patient clothes, the product is not limited thereto.

Preferably, the product may be building materials for air purification such as paints, wallpapers, blinds, sidewalk blocks, median separators, artificial turf, artificial turf filler, elastic pavement materials, flooring materials, asphalt, concrete, and elastic mats.

The present invention relates to a method for preparing an inorganic oxide-based photocatalyst, and the method for preparing an inorganic oxide-based photocatalyst, according to an embodiment of the present invention, may include the steps of: preparing an inorganic oxide; forming a ferrocene layer on the inorganic oxide; and forming a ferrocene-derived iron oxide layer by performing a heat treatment process after the step of forming the ferrocene layer.

The step of preparing an inorganic oxide is a step of preparing an inorganic oxide dispersion or applying the inorganic oxide to a substrate, the dispersion may be applied to a water-based solvent, an oil-based solvent, or a mixture thereof, and the substrate may be a silicon substrate, a wafer, a glass substrate, a semiconductor substrate, a metal substrate, or the like. The inorganic oxide may be applied by spin coating, roll coating, spray coating, dip coating, flow coating, doctor blade coating, etc.

In the step of forming the ferrocene layer, the ferrocene layer may be formed by using a wet-type coating method, a sputtering method, or a deposition method. The ferrocene layer may be formed by using a deposition method of preferably atomic layer deposition (ALD), temperature-regulated chemical vapor deposition (TR-CVD), or the like, more preferably TR-CVD. When applying TR-CVD, the amount of ferrocene is adjusted so that the amount of an iron oxide deposited on the inorganic oxide may be easily adjusted, the preparation process of the photocatalyst may be simplified, and the photocatalyst may be efficiently provided.

The step of forming the ferrocene layer may be carried out at room temperature to 120° C., preferably 40 to 100° C., and more preferably 60 to 100° C. Namely, the step of forming the ferrocene layer may be carried out at 60 to 100° C. in order to induce deposition by the vaporization process of ferrocene when applying TR-CVD.

The step of forming the ferrocene layer is carried out in an air or oxygen atmosphere under atmospheric conditions, and an inert gas may be further included.

The step of forming the ferrocene layer may form the ferrocene layer including 0.01 to 20 wt % of ferrocene compared to the inorganic oxide.

According to an embodiment of the present invention, the step of forming the ferrocene-derived iron oxide layer may partially or completely oxidize the ferrocene layer into an iron oxide and may remove impurities such as carbon residues by performing a heat treatment process on the ferrocene layer.

The step of forming the ferrocene-derived iron oxide layer may include performing a heat treatment process in two or more steps at a temperature of 50 to 900° C., or 100 to 800° C.

For example, the step of forming the ferrocene-derived iron oxide layer may include the steps of performing a first heat treatment process at a temperature of 100 to 300° C., and performing a second heat treatment process at a temperature of 300 to 900° C., in which the steps may each include performing the heat treatment process at different temperatures. The steps may be each carried out for 1 minute to 20 hours, and carried out in an air or inert gas atmosphere containing air, 20% or more of oxygen, or 40% or more of oxygen.

In other words, the step of performing the first heat treatment process may be an annealing process for the iron oxide deposition that converts ferrocene into an iron oxide by the reaction of ferrocene with oxygen. The step of performing the second heat treatment process is a post-heat treatment step carried out after the first heat treatment step, and may be an annealing process that improves activity and performance of the photocatalyst by removing impurities such as carbides.

Hereinafter, the present invention will be described in more detail with reference to Examples and Comparative Example.

However, the following Examples are illustrative only, and the contents of the present invention are not limited thereto.

### EXAMPLE 1

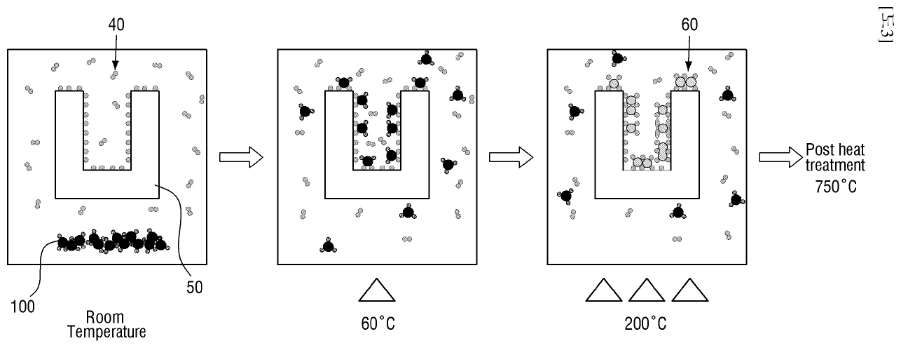

A photocatalyst (Fe—TiO₂) in which nano-sized iron oxide particles were deposited on TiO₂ by using a temperature-regulated chemical vapor deposition (TR-CVD) reactor of FIG. 2 and utilizing TR-CVD shown in FIG. 3. More specifically, 0.02 g of ferrocene, i.e., a precursor of iron, was put in a container made of quartz, and then placed on the inner bottom of a reactor surrounded by a heating band and made of stainless steel. After putting 3 g of TiO₂ (TiO₂, P-25, Evonik, particle size: 25 nm) in a container made of stainless steel wire mesh, and placing 3 g of TiO₂ put in the container on the inner central portion of the reactor, the reactor was sealed using a polyimide tape. Ferrocene was converted into an iron oxide by proceeding with the deposition process of ferrocene as a TR-CVD vaporization process at a reactor temperature of 60° C. for 2 hours, and then increasing the temperature to 200° C., thereby maintaining the temperature for 12 hours.

Subsequently, a photocatalyst (or expressed as Fe—TiO₂) of an iron oxide-TiO₂ hybrid nanostructure was finally prepared by taking out TiO₂, and performing an additional heat treatment process on TiO₂ at 750° C. in a dry air gas atmosphere for 2 hours. The content of iron deposited on TiO₂ under corresponding conditions was about 0.09 wt %.

### EXAMPLE 2

A photocatalyst of an iron oxide-TiO₂ hybrid nanostructure was prepared in the same manner as in Example 1 except that 0.05 g of ferrocene as an iron precursor was applied. The content of iron deposited on TiO₂ under corresponding conditions was about 0.13 wt %.

### EXAMPLE 3

A photocatalyst of an iron oxide-TiO₂ hybrid nanostructure was prepared in the same manner as in Example 1 except that 0.1 g of ferrocene as an iron precursor was applied. The content of iron deposited on TiO₂ under corresponding conditions was about 0.65 wt %.

### EXAMPLE 4

A photocatalyst of an iron oxide-TiO₂ hybrid nanostructure was prepared in the same manner as in Example 1 except that 0.3 g of ferrocene as an iron precursor was applied. The content of iron deposited on TiO₂ under corresponding conditions was about 1.81 wt %.

After comparing transparency and color of the prepared photocatalyst (Fe—TiO₂) with those of a TiO₂ photocatalyst coated with a general iron oxide, the comparison results are shown in FIG. 4. When checking FIG. 4, it may be confirmed that a photocatalyst (Fe—TiO₂) coated with a ferrocene-derived iron oxide has a transparent and light yellow color compared to a photocatalyst (Fe₂O₃-TiO₂) coated with an iron oxide (Fe₂O₃).

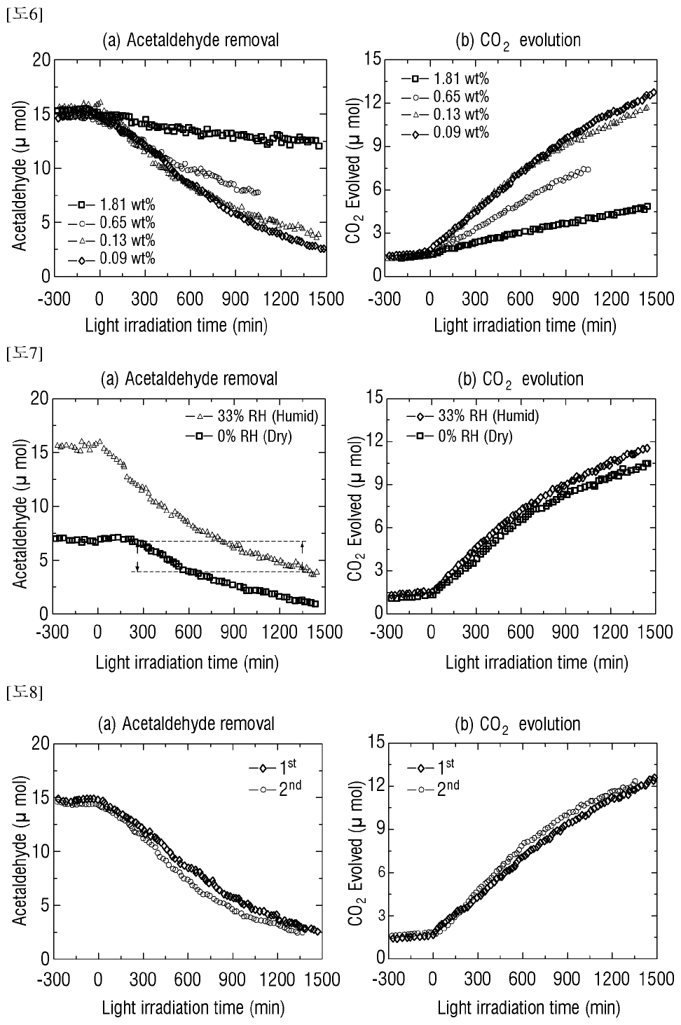

After measuring TEM images (images measured by a transmission electron microscope) of the prepared photocatalysts (Fe—TiO₂), measurement results are shown in FIG. 5. FIG. 5 shows that as the iron content decreases, the size of iron oxide particles deposited on the surface of Fe—TiO₂ decreases.

After measuring specific surface areas (BET) and BJH average pore sizes through nitrogen adsorption analysis of the prepared photocatalysts (Fe—TiO₂), measurement results are shown in Table 1.

TABLE 1

| | 0.13 wt % | 0.65 wt % | 1.81 wt % |
|---|---|---|---|
| | Fe—TiO₂ | Fe—TiO₂ | Fe—TiO₂ |
| BET surface area (m²/g) | 11.6259 | 10.3426 | 8.3939 |
| BJH adsorption average | 13.2 | 12.5 | 13.9 |
| pore size (nm) | | | |

When checking Table 1, it may be confirmed that specific surface areas and average pore sizes are not greatly changed although the iron content of Fe—TiO₂ varies, and it may be confirmed that mesopores of Fe—TiO₂ are formed.

### Evaluation Example 1

After putting the photocatalyst (Fe—TiO₂) of Example 1 in a batch reactor with a volume of 5.3 L of which the top surface was formed of quartz glass, photolysis properties of acetaldehyde were analyzed by irradiating a visible light region with a white LED under conditions including an initial acetaldehyde concentration of 66 ppm, a dry air atmosphere (relative humidity of up to 33%, and a total pressure of 760 torr), and room temperature. Acetaldehyde within the reactor was periodically measured using gas chromatography. The results are shown in FIG. 6.

FIG. 6 is graphs showing (a) change in the mole number of acetaldehyde according to the irradiation time of visible light (white light) under 33% humidity conditions, and (b) change in the mole number of carbon dioxide generated as a result of the photolysis reaction of acetaldehyde. When checking FIG. 6, it may be confirmed that the photolysis of acetaldehyde is performed by the photocatalytic activity due to the irradiation of visible light (white light) in the photocatalysts (Fe—TiO₂) prepared in Examples, and it may be confirmed that the visible light has the greatest decomposition efficiency at a ferrocene deposition amount of 0.09 wt %. Further, it may be confirmed that as the iron content decreases, the acetaldehyde photolysis rate of Fe—TiO₂ increases.

### Evaluation Example 2

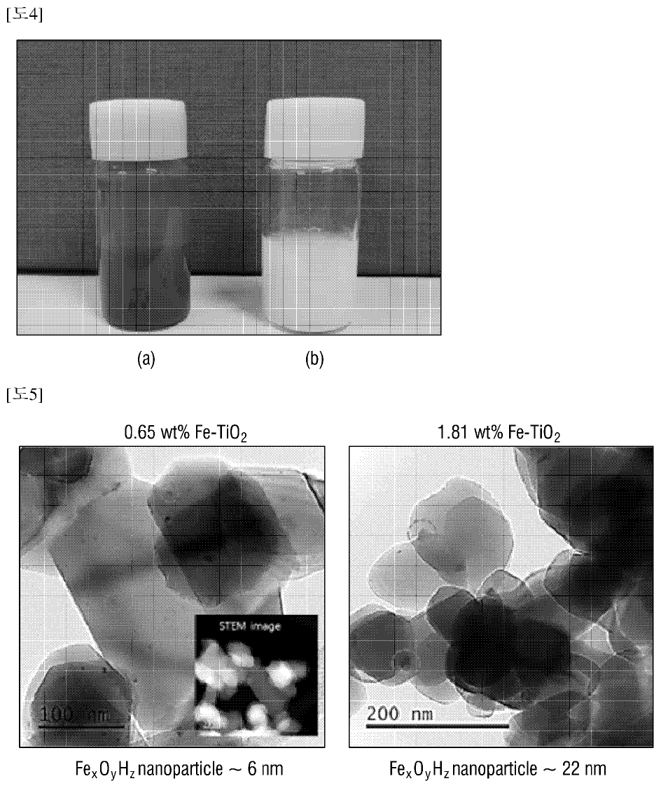

Photolysis properties of acetaldehyde were analyzed in the same manner as in Evaluation Example 1 in dry conditions without humidity and in humidity conditions with a relative humidity of up to 33% respectively by using a photocatalyst (Fe—TiO₂) with a ferrocene deposition amount of 0.13 wt %. Acetaldehyde and carbon dioxide within the reactor were periodically measured using gas chromatography. The results are shown in FIG. 7 and FIG. 8.

FIG. 7 is graphs showing (a) change in the mole number of acetaldehyde according to the irradiation time of visible light when conducting an acetaldehyde photolysis experiment under dry conditions and 33% humidity conditions, and (b) change in the mole number of carbon dioxide generated as a result of the photolysis reaction of acetaldehyde. When checking FIG. 7, it may be seen that slopes of two graphs are similarly shown in the same acetaldehyde concentration section indicated by the dotted line, but the acetaldehyde photolysis activity in visible light irradiation is similarly maintained regardless of the presence or absence of humidity.

Further, it may be confirmed that the generation of carbon dioxide is increased depending on the light irradiation time, i.e., carbon dioxide is generated by the complete oxidation of acetaldehyde.

FIG. 8 is graphs showing (a) change in the mole number of acetaldehyde according to the irradiation time of visible light when repeatedly utilizing acetaldehyde in an acetaldehyde photolysis experiment under 33% humidity conditions, and (b) change in the mole number of carbon dioxide generated as a result of the photolysis reaction of acetaldehyde, and it may be confirmed in FIG. 8 that high photocatalytic activity is maintained even in the repeated photolysis experiment.

Comprehensively, an iron oxide-deposited TiO₂ (hereinafter, referred to as Fe—TiO₂) was utilized in the photolysis experiment of acetaldehyde, i.e., one of the representative volatile organic compounds, and acetaldehyde photolysis activities of Fe—TiO₂ depending on contents of the iron oxide were compared in the present invention. As a result, when the iron content is as low as about 0.09 wt %, the photolysis activity of acetaldehyde of Fe—TiO₂ is the highest, and about 70% of the initial acetaldehyde concentration (about 95 mol ppm) is decreased within 20 hours. In addition, it is confirmed that, although the activity of a photocatalyst is generally greatly affected by the humidity, Fe—TiO₂ prepared in the present invention exhibits similar catalytic activities in drying conditions and humidity conditions so that the photocatalyst activity is not sensitive to the humidity. As a result of conducting a nitrogen adsorption experiment of Fe—TiO₂ with various iron contents, it is confirmed that the iron contents do not greatly affect the total specific surface area of the photocatalyst. Moreover, when checking that the photocatalytic activity of Fe—TiO₂ is greatly affected by the iron content, it may be seen that the electronic structure of an interface between deposited iron oxide nanoparticles and TiO₂ is more important than the surface structure in the activity of the photocatalyst. Further, it may be confirmed through a transmission electron microscope that as the iron content decreases, the size of iron oxide particles existing on the surface decreases, and the photocatalytic activity may be increased when iron oxide particles with a size level of 1 to 3 nanometers are deposited. Judging from the analysis results, when iron oxide nanoparticles with a very small size are contained in an amount of about 0.09 wt %, Fe—TiO₂ is capable of rapidly decomposing acetaldehyde by absorbing light of the visible light region and separating pairs of electrons/holes most efficiently to react the separated pairs of electrons/holes with oxygen/water, thereby producing radicals. Meanwhile, when a target organic matter is not completely oxidized, but partially oxidized, and remains on the surface of the photocatalyst to block the active site, the activity of the photocatalyst decreases, and this is pointed out as one of the biggest problems of the photocatalyst. However, it is confirmed that the catalytic activity is equally maintained even in the repeated acetaldehyde photolysis experiment of Fe—TiO₂ prepared in the present invention, and there is also no problem of deteriorating the catalytic activity accordingly.

Although the above-mentioned Examples have been described by limited Examples and drawings, those skilled in the art may apply various modifications and alterations from the above-mentioned description. For example, appropriate results can be achieved although described techniques are carried out in a different order from a described method, and/or described elements are combined or mixed in a different form from the described method, or replaced or substituted with other elements or equivalents. Therefore, other embodiments, other Examples, and equivalents to patent claims belong to the scope of the patent claims to be described later.

1. An inorganic oxide-based photocatalyst comprising:
an inorganic oxide; and
a ferrocene-derived iron oxide layer formed on the inorganic oxide.

2. The inorganic oxide-based photocatalyst of claim 1, wherein iron is contained in the ferrocene-derived iron oxide layer in an amount of 0.001 to 10 wt % compared to the inorganic oxide.

3. The inorganic oxide-based photocatalyst of claim 1, wherein the ferrocene-derived iron oxide layer is obtained by performing a heat treatment process on ferrocene deposited on the inorganic oxide.

4. The inorganic oxide-based photocatalyst of claim 1, wherein the inorganic oxide includes at least one selected from the group consisting of oxides including at least one of Ti, Zn, Al, and Sn.

5. The inorganic oxide-based photocatalyst of claim 1, wherein the inorganic oxide includes at least one selected from the group consisting of a bead form, a powder form, a rod form, a wire form, a needle form, and a fiber form, and the inorganic oxide has a size of 1 nm to 500 μm.

6. The inorganic oxide-based photocatalyst of claim 1, wherein the inorganic oxide-based photocatalyst has photoactivity in a visible light region of 400 nm or more.

7. The inorganic oxide-based photocatalyst of claim 1, wherein the inorganic oxide-based photocatalyst has photoactivity in a dry condition of 30% or less humidity.

8. The inorganic oxide-based photocatalyst of claim 1, wherein the ferrocene-derived iron oxide includes one or more of compounds represented by the following Chemical Formula 1:
FeₓO_{Y}H_{Z} [Chemical Formula 1]
(X, Y, and Z are each selected from 0 to 3, and X and Y are not 0).

9. The inorganic oxide-based photocatalyst of claim 1, wherein the inorganic oxide-based photocatalyst has a specific surface area of 5 (m²/g) or more and an average pore size of 50 nm or less.

10. A photocatalyst composition comprising the inorganic oxide-based photocatalyst of claim 1, wherein the photocatalyst is contained in an amount of 0.01 to 99 wt % in the photocatalyst composition.

11. A product having a photolysis function, the product comprising the inorganic oxide-based photocatalyst of claim 1.

12. The product of claim 11, wherein the product is a molded body in which the inorganic oxide-based photocatalyst is coated on a substrate, or which includes the inorganic oxide-based photocatalyst.

13. The product of claim 11, wherein the product is applied to building materials for air purification having a function of photolyzing acidic gases and organic substances.

14. The product of claim 13, wherein the building materials for air purification is one or more of paints, wallpapers, blinds, sidewalk blocks, median separators, artificial turf, artificial turf filler, elastic pavement materials, flooring materials, asphalt, concrete, and elastic mats.

15. A method for preparing an inorganic oxide-based photocatalyst, the method comprising steps of:
preparing an inorganic oxide;
forming a ferrocene layer on the inorganic oxide; and
forming a ferrocene-derived iron oxide layer by performing a heat treatment process after the step of forming the ferrocene layer.

16. The method of claim 15, wherein the step of forming the ferrocene layer is performed by using a wet coating method, a sputtering method, or a deposition method, the step of forming the ferrocene layer is carried out at room temperature to 120° C., and the ferrocene layer includes 0.001 to 20 wt % of ferrocene compared to the inorganic oxide.

17. The method of claim 15, wherein the step of forming the ferrocene layer is forming a ferrocene deposition layer using temperature-regulated chemical vapor deposition (TR-CVD).

18. The method of claim 15, wherein the step of forming the ferrocene-derived iron oxide layer includes steps of: performing a first heat treatment process at a temperature of 100 to 300° C.; and performing a second heat treatment process at a temperature of 300 to 900° C., wherein the steps each include performing the heat treatment process at different temperatures.
